(19) United States
(12) Patent Application Publication
(10) Pub. No.: US 2022/0202623 A1
(43) Pub. Date: Jun. 30, 2022

(54) Dressing

(71) Applicant: Tex-Ray Industrial Co., Ltd., Tainan City (TW)

(72) Inventors: Wan-Kuei Yao, Taoyuan City (TW); Anderson Kenny, New Taipei City (CN); Dyson Philip, New Taipei City (TW)

(73) Assignee: Tex-Ray Industrial Co., Ltd.

(21) Appl. No.: 17/195,140

(22) Filed: Mar. 8, 2021

(30) Foreign Application Priority Data
Dec. 24, 2020 (TW) 109145976

(51) Int. Cl. A61F 13/02; A61L 15/22

(52) U.S. Cl. CPC A61F 13/0223; A61F 2013/00251; A61L 15/22

(57) ABSTRACT

Provided is a dressing, including: a skin-friendly layer, a fluid transport structure, and an upper cover. The skin-friendly layer has first extraction guide holes and first injection guide holes. The fluid transport structure, formed on the skin-friendly layer, has an extraction guide groove, second extraction guide holes, an injection guide groove and second injection guide holes, wherein the extraction guide groove separates from the injection guide groove, the second extraction guide holes are formed in the extraction guide groove and communicate with the first extraction guide holes, and the second injection guide holes are formed in the injection guide groove and communicate with the first injection guide holes. The upper cover, formed on the fluid transport structure, has an extraction hole communicating with the extraction guide groove and the second extraction guide holes, and an injection hole communicating with the injection guide groove and the second injection guide holes.

### CROSS-REFERENCE TO RELATED APPLICATION

This application claims the priority of Taiwanese patent application No. 109145976, filed on Dec. 24, 2020, which is incorporated herewith by reference.

### BACKGROUND OF THE INVENTION

### 1. Field of the Invention

The present invention relates generally to a dressing, and more particularly, to a dressing for negative pressure wound therapy.

### 2. The Prior Arts

Nowadays, the medical field develops auxiliary negative pressure wound therapy (NPWT) for complex wounds, surgery, chronic diseases, burns and scalds. It uses a dressing connected to a suction device to cover open wounds and regulates the suction device to draw out the air and the tissue fluid discharged from the wound between the wound and the dressing to form a negative pressure airtight environment between the dressing and the open wound, which can avoid infection, reduce dressing changes and promote wound healing.

Negative pressure wound therapy mainly uses porous materials, such as foam, to make dressings. The pores of the dressing can absorb wound tissue fluid, and the suction device draws out the wound tissue fluid absorbed by the dressing through a suction tube. Because the wound tissue fluid and new granulation tissue easily block the pores of the dressing, the suction device cannot completely extract the wound tissue fluid in the dressing, which affects the negative pressure effect. Therefore, the dressing must be replaced after several days of use, and changing the dressing may destroy the new granulation tissue.

At present, there is a negative pressure wound therapy combined with perfusion. The method is to set a negative pressure time period, inject cleaning or drug solution into the dressing and wound during the negative pressure release, and then start the suction device to extract the cleaning or drug solution. In the process of injecting the solution, the adhesion between the dressing and the wound may be damaged, and the subsequent negative pressure suction may be affected. The treatment staff often needs to adjust the pressure of the suction device, the position of the dressing or change the dressing. The operation is difficult and may reduce the effect of negative pressure therapy. How to solve the problems of the existing dressing, reduce the operating difficulty of negative pressure wound therapy, and improve the effect of negative pressure therapy are the objectives of developing the present invention.

### SUMMARY OF THE INVENTION

To achieve the aforementioned objectives, the present invention provides a dressing, comprising: a skin-friendly layer, a fluid transport structure, and an upper cover. The skin-friendly layer has a plurality of first extraction guide holes and a plurality of first injection guide holes. The fluid transport structure is formed on the skin-friendly layer and has an extraction guide groove, a plurality of second extraction guide holes, an injection guide groove and a plurality of second injection guide holes, wherein the extraction guide groove and the injection guide groove are not connected, the second extraction guide holes are formed in the extraction guide groove and communicate with the first extraction guide holes, and the second injection guide holes are formed in the injection guide groove and communicate with the first injection guide holes. The upper cover is formed on the fluid transport structure and has an extraction hole and an injection hole. The extraction hole communicates with the extraction guide groove and the second extraction guide holes, and the injection hole communicates with the injection guide groove and the second injection guide holes.

In a preferred embodiment, the skin-friendly layer forms a convex portion and a sealing ring on a side opposite to the fluid transport structure, the sealing ring surrounds the convex portion, and the convex portion is adapted to fit a wound.

In a preferred embodiment, the skin-friendly layer forms a plurality of partition walls on a side next to the fluid transport structure, and the partition walls respectively separate the first extraction guide holes from the first filling guide holes.

In a preferred embodiment, the skin-friendly layer is made of silicone rubber or polyurethane.

In a preferred embodiment, the skin-friendly layer has a thickness between 1 and 2 mm.

In a preferred embodiment, the fluid transport structure includes an extraction transport layer and an injection transport layer, the extraction transport layer has the extraction guide groove, the second extraction guide holes, and a third injection guide hole, and the injection transport layer has the injection guide groove, the second injection guide holes and a third extraction guide hole, the third injection guide hole communicates with the injection guide groove, and the third extraction guide hole communicates with the extraction guide groove.

In a preferred embodiment, the extraction transport layer is sandwiched between the injection transport layer and the upper cover.

In a preferred embodiment, the dressing further includes a flow channel layer sandwiched between the skin-friendly layer and the fluid transport structure; the flow channel layer has a plurality of partition walls and a plurality of flow channels arranged in a staggered arrangement, and the partition walls respectively separate the first extraction guide holes and the first injection guide holes, and the flow channel is respectively connected with the first extraction guide holes and the first injection guide holes.

In the dressing of the present invention, the skin-friendly layer, the fluid transport structure and the upper cover are provided with guide grooves and guide holes for separating the extraction and injection of the fluid so that the negative pressure wound therapy can perform fluid extraction, drug delivery and cleaning separately or simultaneously. The drug delivery and cleaning will not affect the adhesion between the skin-friendly layer and the wound, which can reduce the difficulty of negative pressure wound therapy, effectively prevent wound infection, promote wound healing, extend dressing replacement period, and greatly enhance the effect of wound treatment.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is an exploded view of a dressing according to an embodiment of the present invention;

FIG. 1A is a side view of the skin-friendly layer of FIG. 1;

FIG. 1B is a side view of the fluid transport structure of FIG. 1;

FIG. 1C is a side view of the top cover of FIG. 1;

FIG. 1D is a side cross-sectional view of section I-I′ in FIG. 1;

FIG. 2A is a top view of the skin-friendly layer of a dressing according to an embodiment of the present invention;

FIG. 2B is a side view of the skin-friendly layer of a dressing according to an embodiment of the present invention;

FIG. 3 is an exploded view of the dressing of another embodiment of the present invention;

FIG. 3A is a side view of the skin-friendly layer of FIG. 3;

FIG. 3B is a side view of the flow channel layer of FIG. 3;

FIG. 3C is a side view of the injection transport of FIG. 3;

FIG. 3D is a side view of the extraction transport layer of FIG. 3; and

FIG. 3E is a side view of the upper cover of FIG. 3.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

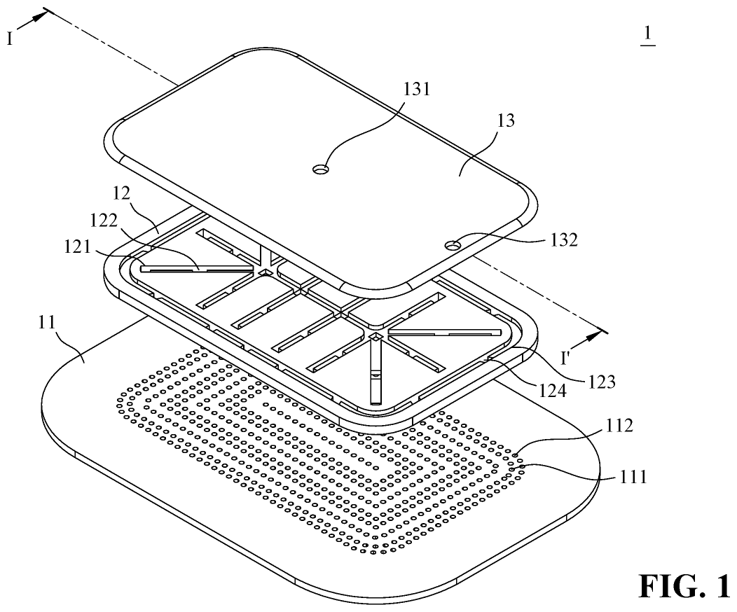

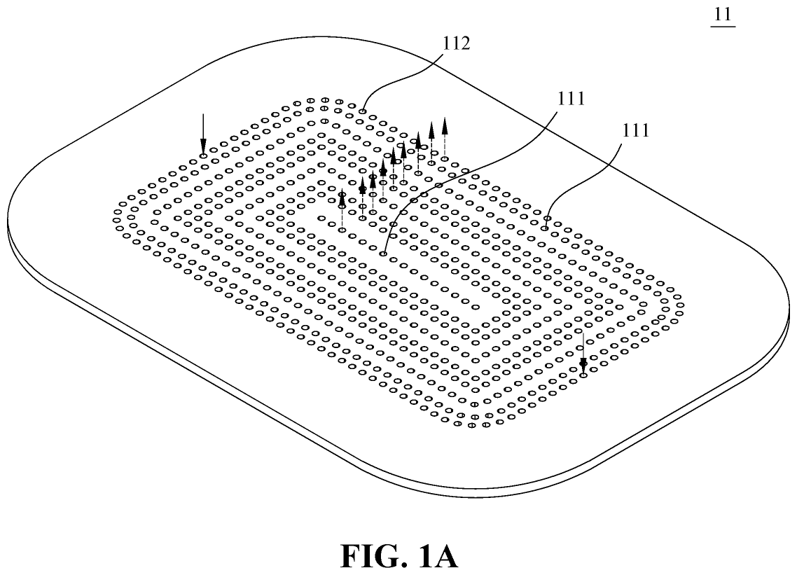

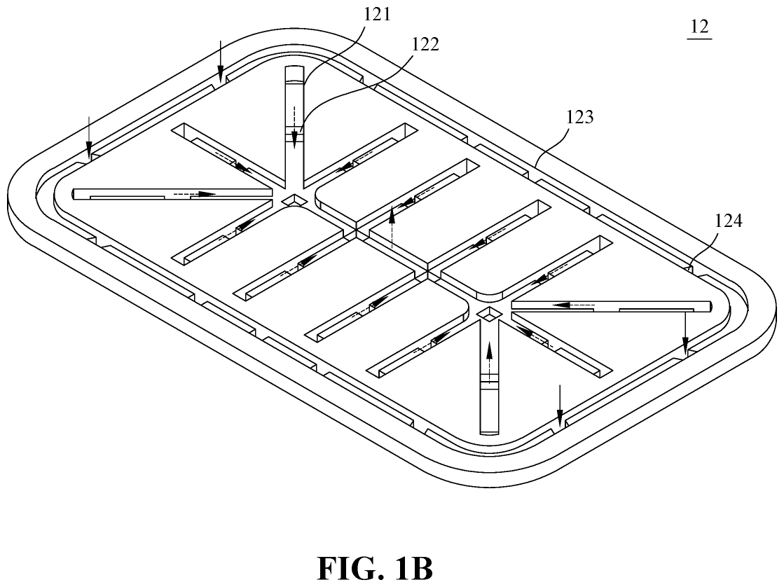

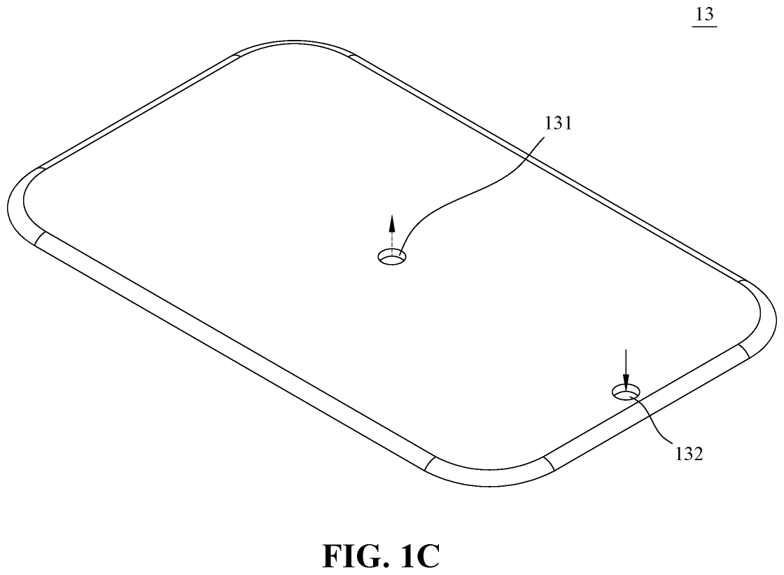

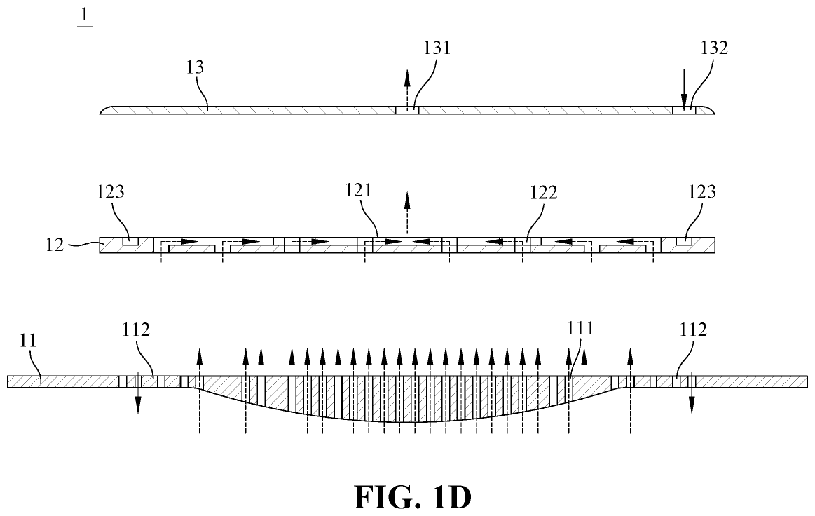

The dressing of the present invention is suitable for negative pressure suction and medicine infusion cleaning of wounds. FIG. 1 is an exploded view of the dressing according to an embodiment of the present invention. FIG. 1A is a side view of the skin-friendly layer of FIG. 1, FIG. 1A is a side view of the skin-friendly layer of FIG. 1; FIG. 1B is a side view of the fluid transport structure of FIG. 1; FIG. 1C is a side view of the top cover of FIG. 1; and FIG. 1D is a side cross-sectional view of section I-I′ in FIG. 1. As shown in FIGS. 1, 1A, 1B, and 1C, in the present embodiment, the dressing 1 includes: a skin-friendly layer 11, a fluid transport structure 12 and an upper cover 13. The skin-friendly layer 11 has a plurality of first extraction guide holes 111 and a plurality of first injection guide holes 112. The fluid transport structure 12 is formed on the skin-friendly layer 11, and has an extraction guide groove 121, a plurality of second extraction guide holes 122, an injection guide groove 123, and a plurality of second injection guide holes 124, wherein the extraction guide groove 121 and the injection guide groove 123 is not connected, the second extraction guide holes 122 are formed in the extraction guide groove 121 and communicate with the first extraction guide holes 111, and the second injection guide holes 124 are formed in the injection guide groove 123 and communicate with the first injection guide holes 112. The upper cover 13 is formed on the fluid transport structure 12 and has an extraction hole 131 and an injection hole 132. The extraction hole 131 communicates with the extraction guide groove 121 and the second extraction guide holes 122, and the injection hole 132 communicates with the injection guide groove 123 and the second injection guide holes 124.

Medical silicone rubber, polyurethane, polypropylene and other polymer materials with good biocompatibility can be used to mold the skin-friendly layer 11, the fluid transport structure 12 and the upper cover 13, respectively, and the thickness of each layer is between 1 and 2 mm. Then, by stacking and hot pressing the skin-friendly layer 11, the fluid transport structure 12 and the upper cover 13, a dressing 1 of the present invention is formed.

As shown by the dotted arrows in FIGS. 1A, 1B, 1C and 1D, during the negative pressure extraction process, the skin-friendly layer 11 of the dressing 1 is attached to the wound, so that the skin-friendly layer 11 and the wound form a sealed space. A suction tube (not shown) of the suction device is connected to the extraction hole 131 of the upper cover 13, and the injection hole 132 of the upper cover is closed using a plug or a drug delivery tube (not shown). After the suction device is activated, the air and the tissue fluid of the wound in the sealed space pass through the first extraction guide holes 111 of the skin-friendly layer 11, the second extraction guide holes 122 and the extraction guide groove 121 of the fluid transport structure 12, and the extraction hole 131 of the upper cover 13, and flow into the container of the suction device.

As shown by the solid arrows in FIGS. 1A, 1B, 1C and 1D, during the drug delivery process, the skin-friendly layer 11 of the dressing 1 is attached to the wound so that a closed space is formed between the skin-friendly layer 11 and the wound, and then the injection hole 132 of the upper cover 13 is connected with an infusion tube (not shown) of an injection device, and a plug or a suction tube (not shown) is used to close the extraction hole 131 of the upper cover 13. After starting the injection device, the medicine flows in through the injection hole 132 of the upper cover 13, the second injection guide holes 124 and the injection guide groove 123 of the fluid transport structure 12, and the first injection guide holes 112 of the skin-friendly layer 11 to reach the wound.

During the cleaning process, the skin-friendly layer 11 of the dressing 1 is attached to the wound, so that a closed space is formed between the skin-friendly layer 11 and the wound, and the suction tube (not shown) of the suction device is connected to the extraction hole 131 of the upper cover 13, the injection hole 132 of the upper cover 13 is connected with an infusion tube (not shown) of the injection device. After the suction device and the injection device are started at the same time, the cleaning solution passes through the injection hole 132 of the upper cover 13, the second injection guide holes 124 and the injection guide groove 123 of the fluid transport structure 12, and the first injection guide hole 112 of the skin-friendly layer 11, and reaches the wound; the air in the closed space, the wound tissue fluid, and the cleaning solution after cleaning the wound pass through the first extraction guide holes 111 of the skin-friendly layer 11, the second extraction guide holes 122 and the extraction guide groove 121 of the fluid transport structure 12, and the extraction hole 131 of the upper cover 13, and flow into the container of the suction device.

The skin-friendly layer, the fluid transport structure, and the upper cover form guide holes and guide grooves that separate the extraction and injection of the fluid. The dressing of the present invention is used so that the negative pressure wound therapy can perform fluid extraction, drug delivery and cleaning separately or simultaneously. The drug delivery and cleaning will not affect the adhesion between the skin-friendly layer and the wound, which can reduce the difficulty of negative pressure wound therapy, effectively prevent wound infection, promote wound healing, extend dressing replacement period, and greatly enhance the effect of wound treatment.

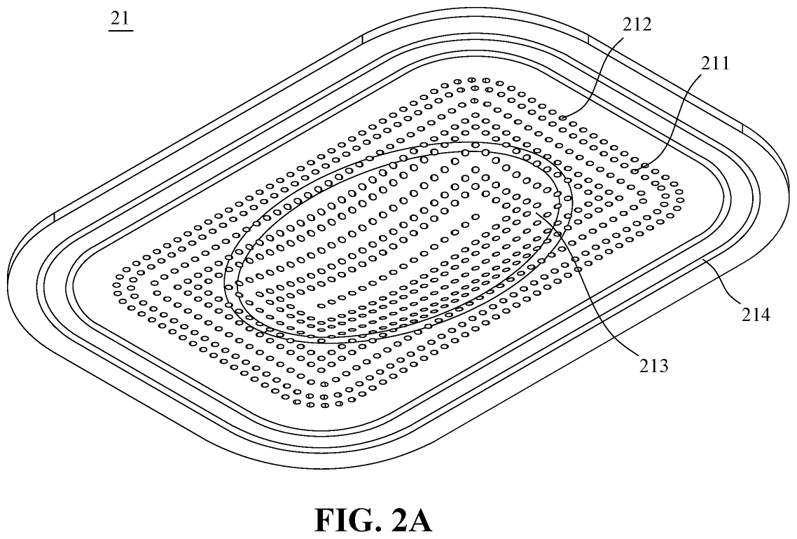

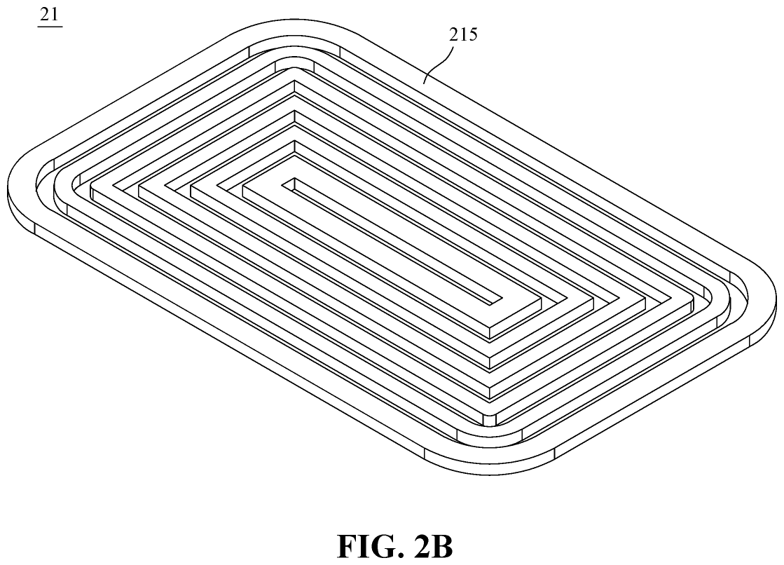

FIG. 2A is a top view of the skin-friendly layer of a dressing according to an embodiment of the present invention; FIG. 2B is a side view of the skin-friendly layer of a dressing according to an embodiment of the present invention. As shown in FIGS. 2A and 2B, in the present embodiment, the skin-friendly layer 21 includes: a plurality of first extraction guide holes 211, a plurality of first injection guide holes 212, a convex portion 213, a plurality of sealing rings 214, and a plurality of partition walls 215. Wherein, the first injection guide holes 212 surround the first extraction guide holes 211, the convex portion 213 and the sealing rings 214 are formed on the side opposite to the fluid transport structure (not shown), and the partition walls 215 are formed the side next to the fluid transport structure. The two sealing rings 214 surround the convex portion 213 and are suitable for fitting to attach to the normal skin around the wound, and the convex portion 213 is suitable for fitting to cover the wound. A double-sided tape (not shown) can be further attached around the sealing ring 214 to improve the adhesion between the skin-friendly layer 21 and the skin around the wound. The partition walls 215 separate the first extraction guide holes 211 from the first injection guide holes 212 to prevent the cleaning solution or medicine from returning to the extraction guide groove and the second extraction guide holes of the fluid transport structure before reaching the wound.

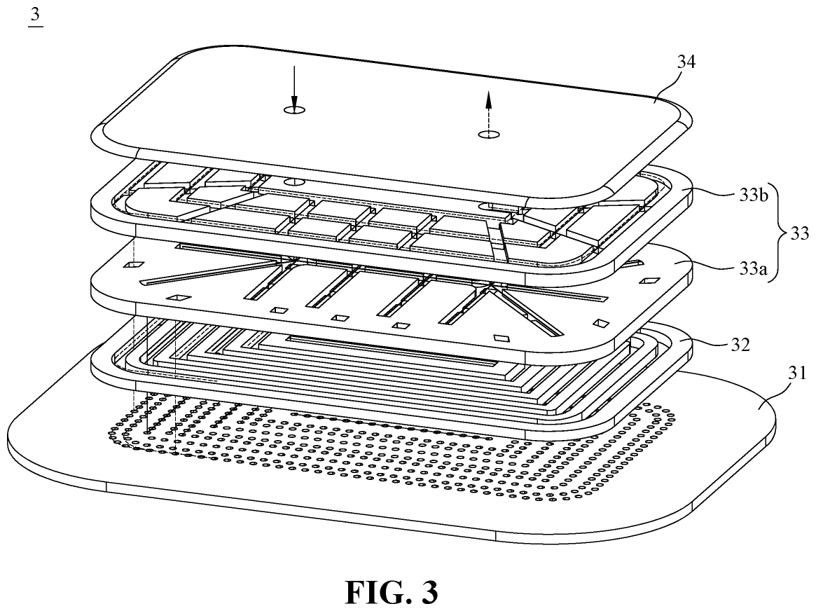

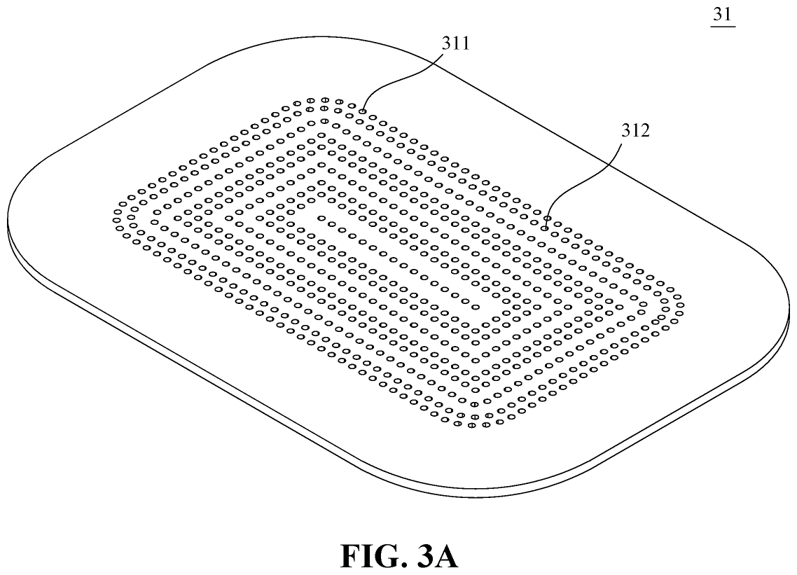

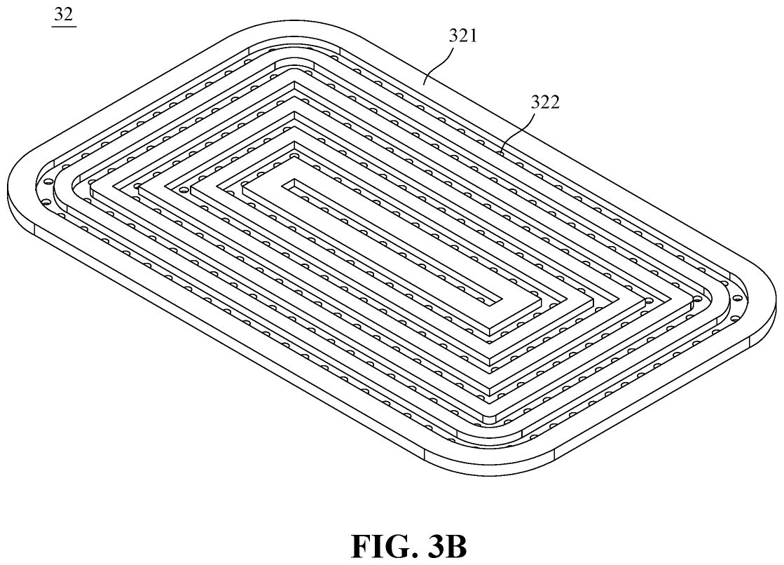

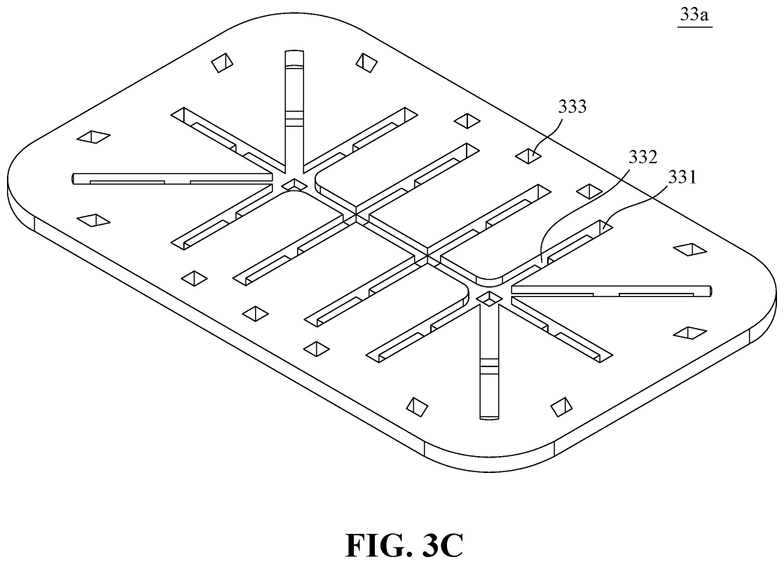

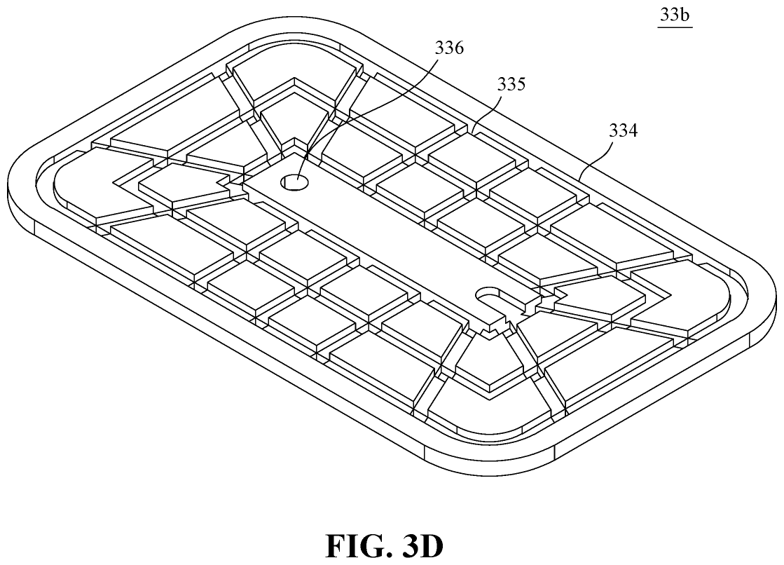

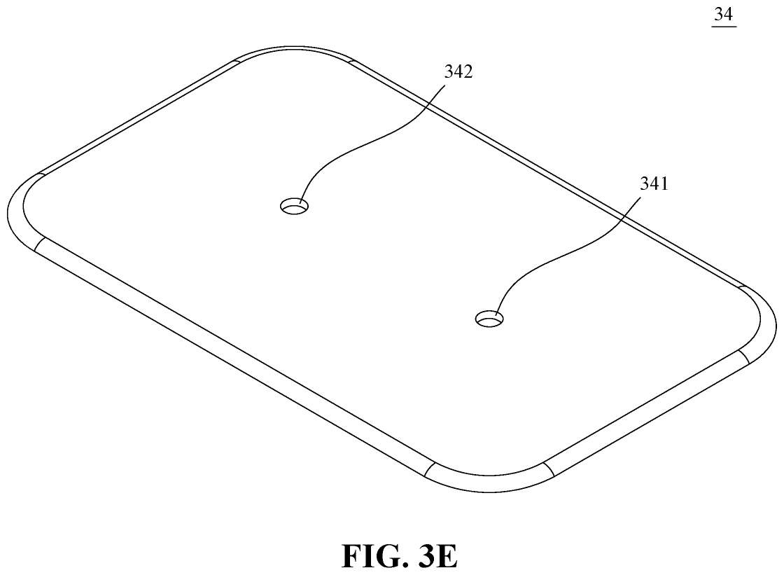

FIG. 3 is an exploded view of the dressing of another embodiment of the present invention; FIG. 3A is a side view of the skin-friendly layer of FIG. 3; FIG. 3B is a side view of the flow channel layer of FIG. 3; FIG. 3C is a side view of the injection transport of FIG. 3; FIG. 3D is a side view of the extraction transport layer of FIG. 3; and FIG. 3E is a side view of the upper cover of FIG. 3. As shown in FIGS. 3, 3A, 3B, 3C, 3D, and 3E, in the present embodiment, the dressing 3 includes: a skin-friendly layer 31, a flow channel layer 32, a fluid transport structure 33, and an upper cover 34, all stacked from bottom to top. The skin-friendly layer 31 has a plurality of first extraction guide holes 311 and a plurality of first injection guide holes 312. The flow channel layer 32 is sandwiched between the skin-friendly layer 31 and the fluid transport structure 33, and has a plurality of partition walls 321 and a plurality of flow channels 322 arranged in a staggered arrangement. The partition walls 321 separate the first extraction guide holes 311 from the first injection guide holes 312, respectively. The flow channels 322 are formed with through holes to respectively communicate with the first extraction guide holes 311 and the first injection guide holes 312. The fluid transport structure 33 includes an injection transport layer 33a and an extraction transport layer 33b. The injection transport layer 33a has an injection guide groove 331, a plurality of second injection guide holes 332, and a plurality of third extraction guide holes 333. The extraction transport layer 33b has an extraction guide groove 334, a plurality of second extraction guide holes 335, and a third injection guide hole 336, the third extraction guide holes 333 communicate with the first extraction guide holes 311 and the second extraction guide holes 335, and the third injection guide hole 336 communicates with the injection guide groove 331 and the second injection guide holes 332. The upper cover 34 is formed on the fluid transport structure 33 and has an extraction hole 341 and an injection hole 342. The extraction hole 341 is connected to the extraction guide groove 334, and the injection hole 342 is connected to the third injection guide hole 336.

As shown by the dotted arrows in FIG. 3, during the negative pressure suction process, the skin-friendly layer 31 of the dressing 3 is attached to the wound, so that a closed space is formed between the skin-friendly layer 31 and the wound, a suction tube (not shown) of a suction device is connected to the extraction hole 341 of the upper cover 34, and the injection hole 342 of the upper cover 34 is closed by a plug or a drug delivery tube (not shown). After the suction device is activated, the air in the enclosed space and the tissue fluid of the wound pass through the first extraction guide holes 311 of the skin-friendly layer 31, the flow channels 322 of the flow channel layer 32, the third extraction guide holes 333 of the injection transport layer 33a, the second extraction guide holes 335 and the extraction guide groove 334 of the extraction transport layer 33b, and the extraction hole 341 of the upper cover 34 to flow into the container of the suction device.

As shown by the solid arrows in FIG. 3, during the drug delivery process, the skin-friendly layer 31 of the dressing 3 is attached to the wound, so that a closed space is formed between the skin-friendly layer 31 and the wound, and then an injection tube (not shown) of an injection device is connected to the injection hole 342 of the upper cover 34, and the extraction hole 341 of the upper cover 34 is closed by a plug or a suction tube (not shown). After starting the injection device, the medicine passes through the injection hole 342 of the upper cover 34, the third injection guide hole 336 of the transport layer 33b, the injection guide groove 331 and the second injection guide holes 332 of the injection transport layer 33a, the flow channels 322 of the flow channel layer 32, and the first injection guide holes 312 of the skin-friendly layer 31 to reach the wound.

During the cleaning process, the skin-friendly layer 31 of the dressing 3 is attached to the wound so that a closed space is formed between the skin-friendly layer 31 and the wound, and the suction tube (not shown) of the suction device is connected to the extraction hole 341 of the upper cover 34, and the injection hole 342 of the upper cover 34 is connected with an injection tube (not shown) of the injection device. After starting the suction device and the injection device at the same time, the cleaning solution passes through the injection hole 342 of the upper cover 34, the third injection guide hole 336 of the extraction transport layer 33b, the injection guide groove 331 the second injection guide holes 332 of the injection transport layer 33a, the flow channels 322 of the flow channel layer 32, and the first injection guide holes 312 of the skin-friendly layer 31 to reach the wound; the air in the closed space, wound tissue fluid, and cleansing solution after cleaning the wound pass through the first extraction guide hole 311 of the skin-friendly layer 31, the flow channels 322 of the flow channel layer 32, the third extraction guide holes 333 of the injection transport layer 33a, the second extraction guide holes 335 and the extraction guide groove 334 of the extraction transport layer 33b, and the extraction hole 341 of the upper cover 34 and flows into the container of the suction device.

In summary, The skin-friendly layer, the fluid transport structure, and the upper cover form guide holes and guide grooves that separate the extraction and injection of the fluid. The dressing of the present invention is used so that the negative pressure wound therapy can perform fluid extraction, drug delivery and cleaning separately or simultaneously. The drug delivery and cleaning will not affect the adhesion between the skin-friendly layer and the wound, which can reduce the difficulty of negative pressure wound therapy, effectively prevent wound infection, promote wound healing, extend dressing replacement period, and greatly enhance the effect of wound treatment.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A dressing, comprising:
a skin-friendly layer, having a plurality of first extraction guide holes and a plurality of first injection guide holes;
a fluid transport structure, formed on the skin-friendly layer and having an extraction guide groove, a plurality of second extraction guide holes, an injection guide groove, and a plurality of second injection guide holes, wherein the extraction guide groove and the injection guide groove being not connected, the second extraction guide holes being formed in the extraction guide groove and communicating with the first extraction guide holes, and the second injection guide holes being formed in the injection guide groove and communicating with the first injection guide holes; and
an upper cover, formed on the fluid transport structure and having an extraction hole and an injection hole; the extraction hole communicating with the extraction guide groove and the second extraction guide holes, and the injection hole communicating with the injection guide groove and the second injection guide holes.

2. The dressing according to claim 1, wherein the skin-friendly layer forms a convex portion and a sealing ring on a side opposite to the fluid transport structure, the sealing ring surrounds the convex portion, and the convex portion is adapted to fit a wound.

3. The dressing according to claim 1, wherein the skin-friendly layer forms a plurality of partition walls on a side next to the fluid transport structure, and the partition walls respectively separate the first extraction guide holes from the first filling guide holes.

4. The dressing according to claim 1, wherein the skin-friendly layer is made of silicone rubber or polyurethane.

5. The dressing according to claim 1, wherein the skin-friendly layer has a thickness between 1 and 2 mm.

6. The dressing according to claim 1, wherein the fluid transport structure comprises an extraction transport layer and an injection transport layer, the extraction transport layer has the extraction guide groove, the second extraction guide holes, and a third injection guide hole, and the injection transport layer has the injection guide groove, the second injection guide holes, and a third extraction guide hole, the third injection guide hole communicates with the injection guide groove, and the third extraction guide hole communicates with the extraction guide groove.

7. The dressing according to claim 6, wherein the extraction transport layer is sandwiched between the injection transport layer and the upper cover.

8. The dressing according to claim 1, wherein the dressing further comprises a flow channel layer sandwiched between the skin-friendly layer and the fluid transport structure; the flow channel layer has a plurality of partition walls and a plurality of flow channels arranged in a staggered arrangement, and the partition walls respectively separate the first extraction guide holes and the first injection guide holes, and the flow channels are respectively connected with the first extraction guide holes and the first injection guide holes.
